(19) United States
(12) Patent Application Publication
(10) Pub. No.: US 2022/0040362 A1
(43) Pub. Date: Feb. 10, 2022

(54) DUAL-MODE SANITZER

(71) Applicant: Neon, LLC, Catharpin, VA (US)

(72) Inventors: Nicholas L. Kulick, Catharpin, VA (US); Michael J. Kulick, Catharpin, VA (US); Kathryn A. Kulick, Catharpin, VA (US)

(21) Appl. No.: 17/386,924

(22) Filed: Jul. 28, 2021

Related U.S. Application Data
(60) Provisional application No. 63/063,492, filed on Aug. 10, 2020.

(51) Int. Cl. A61L 9/20; A61L 2/10

(52) U.S. Cl. CPC A61L 9/20; A61L 2/10; A61L 2209/14; A61L 2209/12; A61L 2202/11

(57) ABSTRACT

A sanitizer may operate in two modes and in its primary mode include a UVC lamp, enclosed by a base with electronic components, a shroud with fan and filter attached, to cover the UVC lamp. The shroud may have inlet holes at one end thereof. When the fan is turned on, air may be drawn in from the exterior through the inlet holes of the shroud. The fan may continue to direct the drawn in air through the filter, across the UVC lamp, and then back to the exterior of the sanitizer. Thus, the air may be filtered of ozone and cleansed of pathogens, even when in the presence of room occupants. To operate in its secondary mode, the sanitizer would have the above-described shroud removed to emit UVC light directly to sanitize both air and surfaces simultaneously in a space without occupants.

### TECHNICAL FIELD

The embodiments described herein pertain generally to ultraviolet germicidal irradiation (UVGI) devices, i.e., ultraviolet C (UVC or UV-C) light devices, for disinfection of air and surfaces.

### BACKGROUND

At the height of the COVID-19 pandemic of 2019-2021, public gatherings were, at best, limited to a handful of participants in places where previously dozens, hundreds, or even thousands were previously permitted; and, at worst, were simply banned due to the extreme transmissibility of SARS-CoV-2.

Gathering places e.g., offices, schools, restaurants, theaters, gyms, etc., slowly re-opened with strict sanitizing and disinfecting protocols to be implemented by both cleaning staff and office workers, students, patrons, and members. The most stringent sanitizing and disinfecting measures were initially taken after-hours, i.e., after the office workers, students, patrons, and members, were required to vacate the premises. Such stringent measures included sanitizing and/or disinfecting every surface, seat, utensil, object, etc., with which a person would come into contact during operational hours, by applying a surface disinfectant and wiping. In addition, or alternatively, the stringent measures also included exposing the interior space of the gathering place to UVC light, which has been proven to deactivate a range of known pathogens including SARS-CoV-2 at the RNA level. As the pandemic progressed, it was learned that COVID-19 spread much more efficiently by aerosolized droplets than by surface contamination.

### SUMMARY

In one example embodiment, a dual-mode sanitizer includes a tower base and a removable shroud. The tower base includes a base in which electronic components may be housed and a UVC lamp. The removable shroud, which may cover the tower base, includes a hollow bottom portion to surround the UVC lamp, and the hollow bottom portion has inlet holes at a lower end thereof; and a top portion to cover the UVC lamp. The top portion includes a fan compartment to house a fan therein and a filter compartment to house a filter therein. The filter compartment may be disposed on top of the fan compartment, and a top portion of the filter compartment may be ventilated to facilitate an exchange of air between the interior and the exterior of the shroud. When the shroud is removed from the tower base, the UVC lamp sanitizes air and surfaces exposed to UVC light radiating therefrom; and when the shroud is in place to cover the tower base, the UVC lamp sanitizes air, that is drawn from the room or space, within the interior of the shroud and then exhausts the air back into the space.

In another example embodiment, a sanitizing method may include activating a UVC light to sanitize air and surfaces that surround the UVC light; turning off the UVC light; covering the UVC light with a shroud that surrounds the UVC light laterally with a UVC-protective material cover; turning on the UVC light; activating the fan to pull air into an enclosure created when the UVC light is covered with the shroud; sanitizing the air within the enclosure by exposing the drawn in air to the UVC light and the carbon filter; and expelling the sanitized air. The filter compartment is disposed over, or next to the fan.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1A shows an example embodiment of a shroud for a sanitizer, in accordance with at least some embodiments described and recited herein;

FIG. 1B shows the example embodiment of the sanitizer of FIG. 1A, uncovered, in accordance with at least some embodiments and examples of deployment described and recited herein;

FIG. 2 illustrates an example embodiment of a shroud covering a sanitizer UVC light unit, in accordance with at least some embodiments described and recited herein; and

FIGS. 3A-3D illustrates progressive installation of a shroud onto a sanitizer, in accordance with at least some embodiments described and recited herein.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not intended to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

For example, in accordance with various embodiments of sanitizer 100, it is contemplated that alternative configurations may include transposing an order of components. Thus, whereas some features may be illustrated and/or described as being towards a bottom of an embodiment, alternative embodiments may contemplate such features being reconfigured to be towards a top of an alternative embodiment. Further, while placement of UVC lamps is in a 360 degree configuration, alternative embodiments may contemplate lamps in any one, two, or three dimensional configuration. While figures herein depict one vertical side view of sanitizer 100 and its components, multiple sides may exist and may or may not be identical to one another, depending upon UVC lamp configuration.

As set forth above, stringent sanitization and disinfecting protocols for gathering places, e.g., offices, schools, restaurants, gyms, etc., may include exposing the interior space thereof to a UVC light. However, utilization of commercial-grade UVC light devices may be cost-prohibitive to some building or establishment owners, school systems, etc., having price tags in the range of $50,000-125,000 (U.S. Dollars). Further, commercial-grade UVC light devices are typically of a size that is impractical for onsite storage. Further still, and perhaps of most significant concern, UVC light devices, particularly those that are commercial-grade, pose health risks to people, animals, plants and vulnerable objects. In particular, the U.S. Food and Drug Administration (FDA) advises of reports of skin and eye burns resulting from human exposure to light radiating or emanating from UVC lamps. Further still, direct emanation UVC devices do not address aerosolized infected droplets transmitted from one person to another in occupied spaces, which may be the most common mode of viral spread.

Accordingly, the embodiments described and recited pertain to a UVC lamp, corresponding base, and corresponding shroud that may be utilized without incurring a burn risk when activated in the presence of people, animals, plants and certain vulnerable objects.

Described and recited herein are devices that utilize ultraviolet germicidal irradiation (UVGI) devices, i.e., ultraviolet C (UVC or UV-C) light devices, for disinfection of air and surfaces. Unless context specifically requires, reference herein will be made to “UVC light” to indicate ultraviolet C (UVC or UV-C).

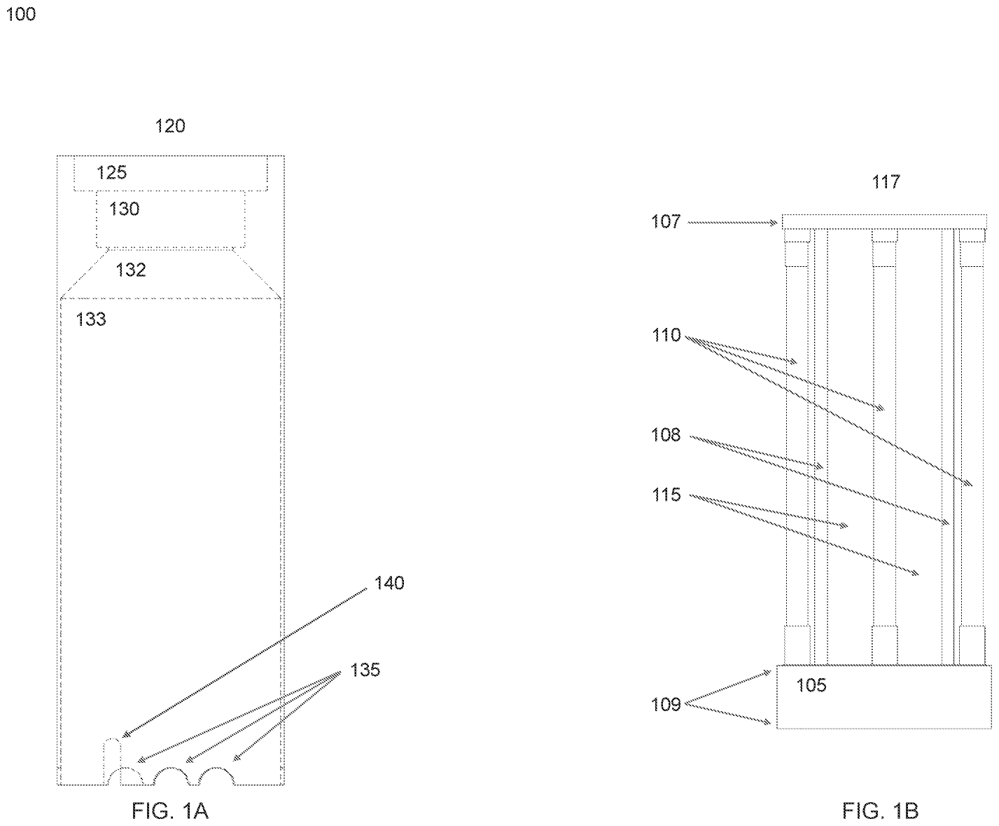

FIG. 1A shows an example of a shroud to cover UVC light unit 117. Shroud 120 includes at least filter compartment 125 and fan compartment 130. Inlet/exhaust holes 135 may be disposed at a bottom portion or, alternatively, a top portion of shroud 120, and may serve as a portal for drawing in air from outside shroud 120 or expelling air outside of shroud 120. Access hole 140 may be provided to grant access to an interior of shroud 120 for, e.g., an electrical cord to provide power to, e.g., electrical components of UVC light unit 117.

FIG. 1B shows the example of sanitizer 100 major component, UVC light unit 117, uncovered by shroud 120, in accordance with various embodiments described and recited herein. As depicted, UVC light unit 117 includes at least tower base 105, one or more ultraviolet-C germicidal lamps 110 and one or more reflectors 115, and structural components: lamp unit cap 107, lamp unit center stile 108, and lamp unit base cap 109.

Tower base 105 may refer to a foundational component of sanitizer 100, to which UVC lamps 110 and reflectors 115 may be permanently or removably attached. In accordance with various embodiments of sanitizer 100, tower base 105 includes a hallowed base in which at least one or more ballasts, wiring, an A/C adaptor, and/or other electrical components for powering at least the one or more UVC lamps may be stowed.

UV-C light unit 117 may include at least one or more UVC lamps 110, one or more reflectors 115, tower base 105 and structural components: lamp unit cap 107, lamp unit center stile 108, and lamp unit base cap 109.

UVC lamp unit cap 107 may be provided to cap UVC light unit 117, may provide access to UVC lamps 110 for removal or replacement, and may be formed of or fabricated from, e.g., PETG plastic, metal or other material that is capable of blocking and otherwise demonstrate resistance to and degradation from UVC light.

UVC lamps 110 may refer to one or more UVC light sources having disinfectant and sanitizing capabilities. The embodiments described and recited herein may utilize UVC lights of various wavelengths, with non-limiting example embodiments of sanitizer 100 including one or more UVC lamps 110 that emit light at a wavelength of 254 nanometers, which is known to be an effective wavelength to kill both airborne and surface pathogens. In accordance with at least some example embodiments, UVC lamps 110 may be electrically connected to tower base 105, drawing power from the ballasts, wiring, A/C adaptor, and other electrical components housed within tower base 105, and may extend upward from tower base 105 to be of sufficient length to ensure a desired length of time of exposure of the passing air to UVC rays. The desired length of time may be measured in fractions of a second.

Reflectors 115 may refer to reflective devices that may be disposed interior to the one or more UVC lamps 110, vertically and/or horizontally, to amplify or intensify the UVC rays radiating horizontally or laterally. That is, any UVC rays directed towards an interior direction relative to tower base 105, and center stile 109, may be redirected outwardly in a horizontal or lateral direction, thus amplifying or intensifying UVC rays that are exposed to surrounding air or surfaces when shroud 120 does not cover UVC light unit 117 or amplifying or intensifying UVC rays that are exposed to air passing along a length of the one or more UVC lamps 110 when shroud 120 is in place to cover UVC light unit 117 and, therefore, the one or more UVC lamps 110 as well.

In at least one alternative embodiment of sanitizer 100, at least some reflectors 115 may be disposed on an interior surface of shroud 120. Thus, any UVC rays directed towards shroud 120 may be redirected inwardly in a horizontal or lateral direction, thus amplifying or intensifying UVC rays that are exposed to air passing along a length of the one or more UVC lamps 110 when shroud 120 is in place to cover UVC lamp unit 117 and, therefore, the one or more UVC lamps 110 as well.

UVC lamp unit center stile 108 may be provided to structurally support the one or more UVC lamps 110 and one or more of reflectors 115. Center stile 108 may be formed of or fabricated from, e.g., PETG plastic, metal or other material that is capable of blocking and otherwise demonstrate resistance to and degradation from UVC light.

UVC lamp unit base cap 109 may be provided to cap tower base 105, thus restraining electrical components housed within. Base cap 109 may be formed of or fabricated from, e.g., PETG plastic, metal or other material that is capable of blocking and otherwise demonstrate resistance to and degradation from UVC light.

Shroud 120 may be provided as a removable component of dual-mode sanitizer 100. Shroud 120 may be molded or otherwise formed so as to be placed over, and therefore cover, UVC light unit 117, which results in the one or more UVC lamps being covered as well. Shroud 120 may refer to a covering made of a PETG (polyethylene terephthalate glycol) plastic or other composite material having UVC protective qualities and characteristics.

In accordance with various embodiments of sanitizer 100, shroud 120 may be battery powered or electrically powered. By at least one example embodiment, shroud 120 may have an electrical cord attached thereto to draw from an AC power source. By at least one other example embodiment, shroud 120 may be configured to electrically connect to a power receptacle corresponding to UVC light unit 117, by which AC power may be drawn.

Shroud 120 may be configured to house and electrically power fan compartment 130 and filter compartment 125.

In accordance with the example configurations of FIGS. 1A and 1B, it will be readily understood that features of shroud 120, fan compartment 130, and filter compartment 125, as generally illustrated and described herein, may be arranged, substituted, combined, separated, and designed in a wide variety of different configurations, all of which are explicitly contemplated herein.

Fan compartment 130 may refer to a housing for an exhaust fan that may be set to a speed sufficient to draw in air from the exterior of sanitizer 100. Fan compartment 130 may be disposed such that blades of one or more fans housed therein rotate, thus producing an airflow in either vertical direction. Further, fan compartment 130 may be configured to accommodate one or more fans housed within to be tilted to various angles to generate airflow of various angles and various intensities.

In accordance with at least one non-limiting example embodiment, one or more fans housed within fan compartment 130 may draw in air from the exterior of sanitizer 100 through the inlet holes at one end of shroud 120, across a length of the one or more UVC lamps 110, and further towards a top of sanitizer 100 so as to be exhausted out to the exterior of the sanitizer 100 through a filter housed in filter compartment 125 and through a ventilated cover of filter compartment 125, with the ventilated cover being on a surface at the end opposite the inlet holes and/or one or more side surfaces of filter compartment 125. The hatching on filter compartment 125 is intended to illustrate the ventilation thereof, without limiting the embodiments herein to having filter side surfaces.

Accordingly, both a bottom surface and a top surface of fan compartment 130 may be ventilated to varying and customizable extents to facilitate the passage of airflow there through. In accordance with at least one other non-limiting example embodiment, the airflow produced by the fan housed within fan compartment 130 may be reversed so as to draw in air from the exterior of sanitizer 100 through the ventilated cover of filter compartment 125, through the one or more filters housed in filter compartment 125, across a length of the one or more UVC lamps 110, and further towards the opposite end of sanitizer 100 from the ventilated cover so as to be exhausted out to the exterior of sanitizer 100 through inlet/exhaust holes 135 of shroud 120. The cover of filter compartment 125 may be ventilated on a top or end surface and/or one or more side surfaces thereof.

An interior structural chamber 132 may be configured or formed within shroud 120 to efficiently funnel air towards fans disposed within fan compartment 130, or in reverse over the one or more UVC lamps 110. Chamber 132 may be fabricated as a portion of shroud 120, formed of or fabricated from, e.g., PETG plastic, metal or other material that is capable of blocking and otherwise demonstrate resistance to and degradation from UVC light.

Another interior structural chamber 133 may be configured or formed within shroud 120 to enclose UVC light unit 117. Chamber 133 may be fabricated as a portion of shroud 120, formed of or fabricated from, e.g., PETG plastic, metal or other material that is capable of blocking and otherwise demonstrate resistance to and degradation from UVC light.

Filter compartment 125 may refer to a housing for one or more carbon filters that is disposed at a top portion of shroud 120. The one or more carbon filters may be disposed horizontally or vertically or at tilted angles there between. Filter compartment 125 may be disposed on top of fan compartment 130, with the bottom surface of filter compartment 125 being disposed atop a top ventilated surface of fan compartment 130, and the top surface of filter compartment 125 being the top surface of shroud 120. Thus, the bottom surface of filter compartment 125 is ventilated to facilitate passage of air relative to fan compartment 130. In accordance with at least one example embodiment of sanitizer 100, the top surface of filter compartment 125 may be ventilated to facilitate passage of air relative to an exterior of sanitizer 100 when shroud 120 is in place to cover UVC light unit 117 and, therefore, the one or more UVC lamps 110; alternatively, or additionally, one or more side surfaces of filter compartment may be ventilated to facilitate passage of air relative to the exterior of sanitizer 100 when shroud 120 is in place to cover UVC light unit 117 and, therefore, the one or more UVC lamps 110.

In accordance with various example embodiments, filter compartment 125 may house one or more carbon filters to filter out ozone from air drawn in to an enclosure created when shroud 120 is in place to cover UVC light unit 117 through the inlet openings at one end of shroud 120. Alternatively, the one or more carbon filters housed in filter compartment 125 filter out ozone air drawn in from the exterior sanitizer 100 through a ventilated surface at the opposite end to the above and/or one or more ventilated side surfaces of filter compartment 125, when the airflow produced by the bidirectional fan housed in fan compartment 130 draws in air from that end of shroud 120.

In accordance with various example embodiments, one or more horizontally disposed filters housed in filter compartment 125 may be configured so that a circumference thereof substantially matches the inner circumstance of shroud 120. In accordance with other example embodiments, one or more vertically disposed filters may be housed in filter compartment 125. Accordingly, air drawn in from the exterior of the device may be fully filtered of ozone prior to being sanitized; in addition, or in the alternative, air sanitized by the one or more UVC lamps 110 may be filtered of ozone prior to being recirculated or emitted to the exterior of sanitizer 100.

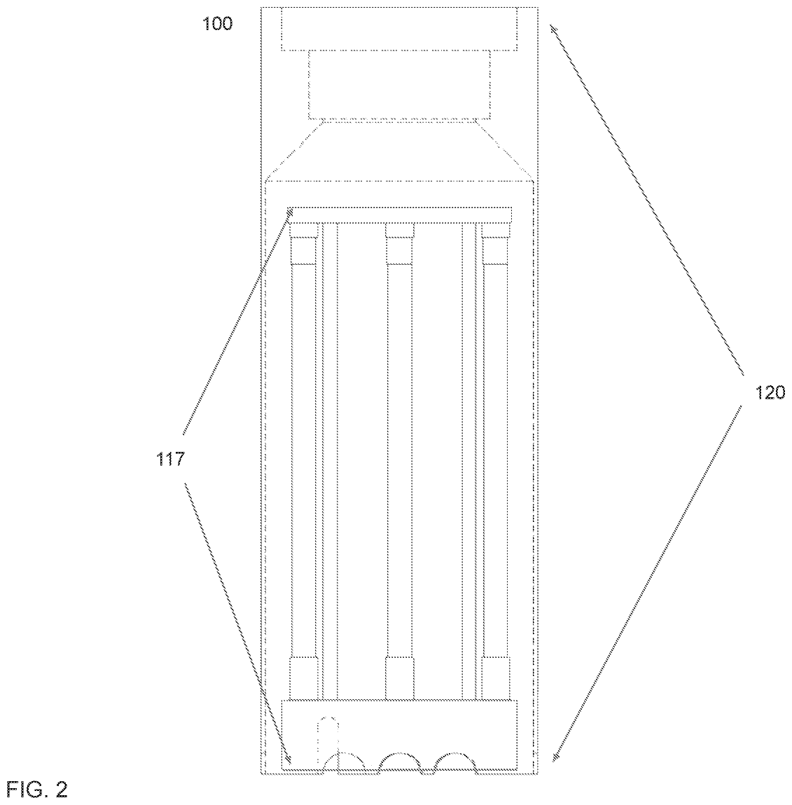

FIG. 2 illustrates an example embodiment of sanitizer 100 with shroud 120 covering light unit 117, in accordance with at least some embodiments described and recited herein. In particular, FIG. 2 provides a transparent view of shroud 120 as it covers UVC light unit 117.

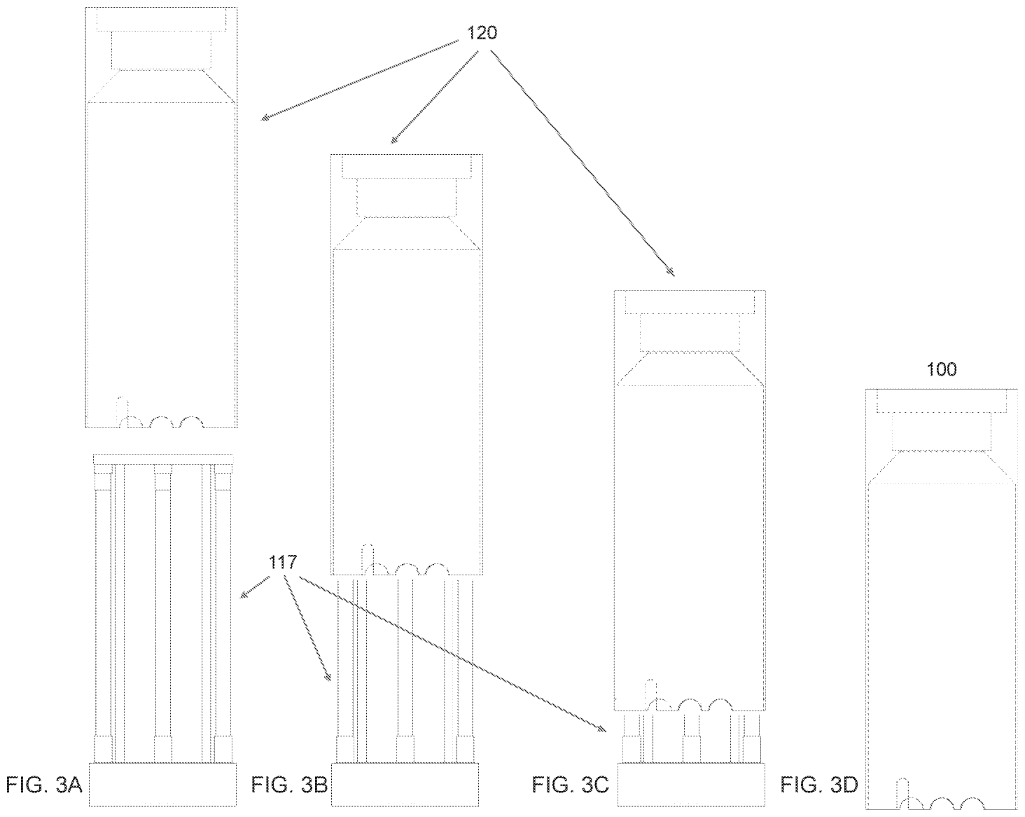

FIGS. 3A-3D illustrate progressive installation of a shroud onto a sanitizer, or UVC light unit 117, in accordance with at least some embodiments described and recited herein.

When shroud 120 is in place to cover tower base 105, as progressively illustrated in FIGS. 3A-3D, and therefore cover the one or more UVC lamps 110, UVC rays do not penetrate any intact surface of shroud 120 so as to protect people, animals or vulnerable objects from UVC exposure who would otherwise be exposed to the UVC rays in the absence of shroud 120. Further, as described below, when shroud 120 is in place to cover UVC light unit 117, one or more fans disposed within fan compartment 130 may direct air drawn in through inlet holes 135 cut or otherwise fashioned at one end of shroud 120 across the one or more UVC lamps 110, through a filter disposed within filter compartment 125, and to the exterior of sanitizer 100 through ventilated end and/or side portions of filter compartment 125.

In the alternative, when shroud 120 is in place to cover tower base 105 and one or more UVC lamps 110, an airflow direction of the one or more fans disposed within fan compartment 130 may be reversed to direct air drawn in through the ventilated end portion of filter component 125 through the filter disposed within filter compartment 125, across the one or more UVC lamps 110, and through the inlet holes 135 at the other end of shroud 120.

Shroud 120 may be molded or otherwise to cover tower base 105 such that side-wall portions of shroud 120 block or otherwise prevent horizontal emanation of UVC rays from the one or more UVC lamps 110. Vertical emanation of UVC rays from the one or more UVC lamps 110 may be blocked or otherwise prevented, in an upward direction, by a top portion of shroud 120, which may include fan compartment 130, on top of which is disposed filter compartment 125. The vertical emanation in a downward direction may be blocked or otherwise prevented by a bottom portion of the tower base 105 end cap on which sanitizer 100 is set.

Shroud 120 may have a series of inlet holes 135 cut or otherwise fashioned at one end. The inlet holes 135 may be provided to allow air to be drawn into an enclosure created when shroud 120 is placed over UVC light unit 117 or the inlet holes 135 may be provided for air to be expelled from the enclosure, depending on the direction of airflow provided by the fan housed in fan compartment 130.

Regardless, inlet holes 135 are provided for the passage of air. However, the inlet holes 135 are also provided in a protective manner. That is, as stated above, the materials from which shroud 120 is made have UVC protective qualities and characteristics, though clearly any holes in such materials are unable to block the passage of UVC rays. Thus, the inlet holes 135 are to be disposed at a level towards an end portion of shroud 120 beyond a stem of any of the one or more UVC lamps 110, and therefore laterally across from a portion of tower base 105, so as to avoid exposing people, animals, plant and vulnerable objects in the vicinity of sanitizer 100 with harmful UVC rays emanating laterally or horizontally from the one or more UVC lamps 110. As an example, as non-limiting configuration examples, the inlet holes 135 may be disposed to be between the base end cap, a surface, e.g., floor or table, on which sanitizer 100 is placed and a top surface of tower base 105 on which stems for the one or more UVC lamps 110 are placed.

Shroud 120 may be disposed, relative to the one or more UVC lamps 110, in a variety of ways, none of which are limiting to the embodiments described or recited herein. As one non-limiting example, shroud 120 may be configured akin to a lampshade that fits over the one or more UVC lamps 110, and secured to either of a top or bottom portion of UVC light unit 117.

As another non-limiting example, shroud 120 may be configured akin to a circular or rectangular shower curtain that may be lifted or lowered to surround the one or more UVC lamps 110.

Regardless of the configuration, shroud 120 is to block at least all lateral emanations of UVC rays from the one or more UVC lamps 110. That is, a bottom portion of shroud 120 may be configured to either substantially fit around a bottom of UVC light unit 117 and its endcap.

In particular, FIG. 2 shows sanitizer 100 with shroud 120 in place over UVC light unit 117 and the one or more UVC lamps 110. In the figure, UVC light unit 117, is shown, in accordance with at least one non-limiting example embodiment, as if shroud 120 is transparent. However, the transparency is for illustrative purposes only, and is not intended to be depicted as a feature of sanitizer 100 or even shroud 120.

As described and recited herein, sanitizer 100 may include a contoured enclosure, i.e., shroud, molded fabricated to closely envelop one or more UVC lamps. The shroud may contain a UVC chamber and electrically houses at least one or more fans and further houses one or more filters.

The surface or walls of the shroud may include one or more air inlet holes. Accordingly, the fan may be positioned to draw air into the unit from the inlet holes at an end opposite to where the fan is positioned in order to establish flow across an entire length of the one or more UVC lamp surface for maximum irradiation. The fan may continue the airflow by drawing air through the filters at an end opposite the one or more UVC lamps, from the air inlet, exhausting it out into the room or space.

The device in its primary mode sanitizes by delivering large concentrated doses of UVC light to air that may be drawn across the one or more germicidal lamps. The device may operate by drawing air, via an intake/exhaust fan, from outside of the shroud thru the inlet holes, and across the germicidal lamps. The sanitized air may then be exhausted, via the fan through the filters back into the room or space. The shroud may envelop the one or more UVC lamps that may be positioned within the UVC chamber, blocking emission of UVC light while the germicidal lamp unit is in operation.

The shroud may be removed manually or may be removed by powered means. Once the shroud is removed, the germicidal lamp(s) may operate independently for direct exposure surface and air sanitization simultaneously, thus performing its secondary mode.

A non-limiting example of intended use is in a classroom occupied by people, including a teacher and students. Capacity and configuration of the room meet current local health and safety department policies. During the school day when teachers and students may occupy the classroom, the device may be positioned, or mounted, on a table, shelf, cart, wall or other structure. Multiple sanitizer units may be present and may be positioned in and around the room or space according to size, configuration and established airflow of the space. Larger rooms or spaces with greater distribution of occupants may have more sanitizer devices present.

Placement of devices may take current, established airflow into account. Prior to occupants entering the room/space, the sanitizer device may be positioned with both major components, the UVC lamp unit and the shroud installed, connected and powered on. The sanitizer device may operate as in the detailed description above to draw air in, disinfect, and exhaust back into the room/space.

Occupants may be safely shielded from effects of exposure to UVC light via the shroud. To disinfect surfaces, the UVC lamp unit may be powered off and the shroud may be powered off in order to filter ozone from the UVC chamber. When occupants leave the room/space, e.g., to an opposite side of solid structures or glass, the shroud may be safely removed automatically or manually. Then the UVC lamp unit may illuminate and disinfect all surfaces and air exposed and in direct line of light emanating from the UVC. The UVC lamp unit may be powered down before occupants return to the room/space. The shroud may be re-installed to proceed with continuous air disinfection.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

We claim:

1. A dual-mode sanitizer, comprising:
a UVC light unit, comprising:
a base in which electronic components are housed, and
a UVC lamp; and
a removable shroud that covers the tower base, the removable shroud comprising:
a hollow portion to surround the UVC lamp, having inlet/exhaust holes at one end thereof,
a center portion to cover the UVC lamp, a portion at the opposite end from the inlet/exhaust holes comprising:
a fan compartment housing a fan therein, and
a filter compartment housing a filter therein, wherein
the filter compartment is disposed on top of the fan compartment, and
a top or side portion of the filter compartment is ventilated;
wherein the UVC lamp sanitizes air and surfaces exposed to UVC light radiating therefrom when the shroud is removed, and
wherein the UVC lamp sanitizes air within an enclosure created when the shroud is in place to cover the tower base.

2. The dual-mode sanitizer of claim 1, wherein, when the shroud is in place to cover the tower base, the fan directs air drawn in through the inlet holes of the shroud across the UVC lamp, through the filter, and to the exterior of the sanitizer through the ventilated portion of the filter compartment.

3. The sanitizer of claim 1, wherein, when the shroud is in place to cover the tower base, the fan directs air drawn in through the portion of the filter compartment through the filter, across the UVC lamp, and through the inlet/exhaust holes of the shroud.

4. The dual-mode sanitizer of claim 1, wherein the shroud is constructed with UVC protection material.

5. The dual-mode sanitizer of claim 1, wherein the shroud is constructed of PETG plastic.

6. The dual-mode sanitizer of claim 1, wherein the filter is a carbon filter.

7. The dual-mode sanitizer of claim 1, wherein the fan compartment and the filter compartment are interchangeable.

8. The dual-mode sanitizer of claim 1, wherein a direction of airflow for the fan is reversible.

9. The dual-mode sanitizer of claim 1, wherein the tower base further comprises reflectors to amplify UVC light radiating outward when the shroud is removed.

10. A sanitizing method, performed by a sanitizer unit, comprising:
activating a UVC light to sanitize surrounding air and surfaces;
turning off the UVC light;
covering the UVC light with a shroud that surrounds the UVC light laterally with a UVC-protective material and a horizontal cover, the horizontal cover comprising:
a fan, and
a filter that is disposed over the fan; and
turning on the UVC light;
activating the fan to draw in air into an enclosure created when the UVC light is covered with the shroud, wherein
the fan causes the air drawn into the enclosure to be sanitized by exposing the air to the UVC light and the carbon filter, and
the fan expels the sanitized air.

11. The sanitizing method of claim 10,
wherein the activated fan pulls air into the enclosure through inlet holes at one end of the shroud,
wherein the sanitizing comprises:
drawing the air in the enclosure across a length of the UVC light, and
forcing the air through the filter; and
wherein the expelling includes blowing the sanitized air through a ventilated portion of the filter.

12. The sanitizing method of claim 10,
wherein the activated fan pulls air into the enclosure through a ventilated cover of the filter at one end of the shroud,
wherein the sanitizing comprises:
drawing the air through the filter, and
blowing the filtered air in the enclosure across a length of the UVC light; and
wherein the expelling includes blowing the sanitized air through exhaust holes at a bottom portion of the shroud.

13. The sanitizing method of claim 10, wherein the UVC light is amplified by reflectors.

14. The sanitizing method of claim 10, wherein the shroud is constructed with a UVC protective material.

15. The sanitizing method of claim 10, wherein the UVC light is emitted at 254 nanometers.

16. A sanitizing method, performed within a shrouded sanitizer unit, comprising:
covering a UVC light with a shroud that surrounds the UVC light laterally with a UVC protective material and a horizontal cover, the horizontal cover comprising:
a fan, and
a filter that is disposed over the fan;
activating the UVC light;
activating the fan to pull air into an enclosure created by the shroud, wherein
the fan draws the air past the activated UVC light to be sanitized,
the fan blows the sanitized air through the filter, and
the fan exhausts the filtered air through a ventilated surface of the shroud; and
sanitizing air and surfaces surrounding the sanitizer unit by removing the shroud.
